(19) United States
(12) Patent Application Publication
(10) Pub. No.: US 2026/0314624 A1
(43) Pub. Date: Oct. 15, 2026

(54) METHOD FOR PRODUCTION OF CELL-CULTURED INGESTION PRODUCTS

(71) Applicant: TEKNOLOGIAN TUTKIMUSKESKUS VTT OY, Espoo (FI)

(72) Inventor: Heiko RISCHER, Espoo (FI)

(21) Appl. No.: 19/104,315

(22) PCT Filed: Aug. 16, 2023
(86) PCT No.: PCT/FI2023/050469

§ 371 Date: Feb. 17, 2025

(30) Foreign Application Priority Data
Aug. 26, 2022 (FI) 20225753

(51) Int. Cl. A23F 5/02; A23F 5/04; A23P 10/28

(52) U.S. Cl. CPC A23F 5/02; A23F 5/04; A23P 10/28

(57) ABSTRACT

The method of the invention for production of a cell-cultured ingestion product comprises the steps of providing parts of a plant and pre-processing the parts in order to obtain a cell culture by cultivation of the cells as a cell suspension in a nutrient medium. The cells are then separated from the nutrient medium and dried. The dried cells are obtained as a powder. The method is mainly characterized by pressing the obtained power into tablets.

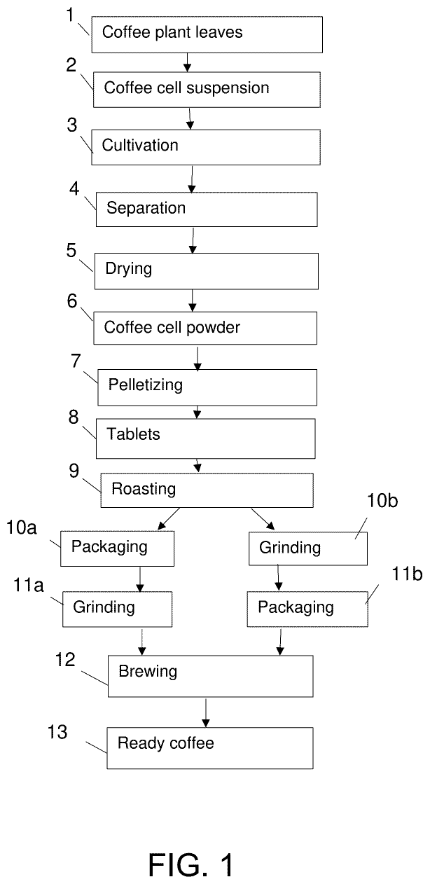

### TECHNICAL FIELD

The invention is concerned with a method for production of cell-cultured products for ingestion including drinks, beverages, foodstuff, and other edible products.

### BACKGROUND

Both cocoa and coffee begin as beans or seeds. Manufacturers grind coffee or cocoa beans for drinks and food ingredients. Cocoa beans, once processed, make the basis for chocolate candy and numerous desserts.

Coffee comes from the seed or bean of the coffee plant, sometimes referred to as the coffee tree and sometimes the coffee shrub being the source for coffee. The coffee plant produces coffee fruits being a type of a stone fruit. The fruit is typically small and green, turning a yellow, deep red or purple shade as it ripens. The coffee bean is housed inside the fruit and technically classified as a seed since it is not a real bean and just resembles one. These bean-like seeds are usually dried, roasted, ground, and brewed to make the coffee.

Coffee and cocoa have some properties in common. However, coffee is made by combining a suitable amount of ground coffee beans and hot water. On the other hand, cocoa is made from roasted, husked, and ground seeds, from which much of the fat has been removed.

Coffee production is the industrial process of converting the raw fruit of the coffee plant into the finished coffee. The coffee fruit or pulp is removed and the seed or bean which is left is then dried. While all raw coffee beans, referred to as green coffee, is processed, the method that is used varies and can have a significant effect on the flavor of roasted and brewed coffee.

Nearly all coffee sold to consumers throughout the world is sold as roasted coffee. Green coffee beans are regular coffee beans that have not been roasted and remain completely raw. Dried bean-like coffee seeds are roasted to varying degrees, depending on the desired coffee flavor in general one of four degrees of roasting: light, medium, medium-dark, and dark. Consumers can also elect to buy unroasted coffee to be roasted at home. Roasted beans are ground and then brewed with near-boiling water to create coffee. Coffee is bitter, darkly colored, slightly acidic, and has a stimulating effect because of its caffeine content

Roasting coffee transforms the chemical and physical properties of green coffee beans into roasted coffee products. The roasting process is what produces the characteristic flavor of coffee by causing the green coffee beans to change in taste. The roasting will turn the coffee beans from a color of light green to brown and forces the hidden oils to come out. The coffee beans will release an intense aroma.

Different varieties of coffee require different roasting routines. This fact makes the roasting process very important. Roasting time depends, among other things, on machinery and technology.

The most common roasting machines are of two basic types: drum and hot-air, although there are others including packed-bed, tangential and centrifugal roasters. Roasters can operate in either batch or continuous modes. Home roasters are also available.

Drum machines consist of horizontal rotating drums that tumble the green coffee beans in a heated environment. The heat source can be supplied by natural gas, liquefied petroleum gas (LPG), electricity, or even wood. The most common employ indirectly heated drums where the heat source is under the drum. Direct-fired roasters are roasters in which a flame contacts the beans inside the drum; very few of these machines are still in operation.

Fluid bed or hot-air roasters force heated air through a screen or perforated plate under the coffee beans with sufficient force to lift the beans. Heat is transferred to the beans as they tumble and circulate within this fluidized bed.

The global coffee production is facing many challenges including land use, climate change and sustainability. At the same time, the global demand for coffee is sharply rising as shown in strongly fluctuating prices. Due to the high demand of coffee, more acreage is required to produce enough coffee beans, leading to deforestation—particularly in sensitive rainforest areas. Because of rising coffee production and consumption worldwide, scientists warn that climate change could also lead to shrinking areas for coffee cultivation.

With increasing demand and numerous sustainability challenges concerning traditional coffee agriculture, there is a pressing need for alternative ways of producing coffee. Cellular agriculture is a promising alternative for the production of plant-based commodities which are conventionally produced by farming. The production of coffee is, however, a complex process. The flavour precursors for coffee in such cellular production are derived from coffee cells that are harvested, dried and roasted to a typical coffee-like flavor.

Attempts have been made by growing coffee in a lab from cell cultures in a bioreactor or steel vessels filled with a nutrient-rich broth. The aim is to produce a brew that smells and tastes like ordinary coffee without growing any coffee plant. In such a process, cell cultures are floating in bioreactors filled with nutrient medium.

The process includes initiating coffee cell cultures, establishing respective cell lines in the laboratory and transferring them to bioreactors to begin producing biomass. After having produced the biomass, drying and roasting processes have to be developed for producing the new type of cell cultured artificial coffee.

The article “Production of Coffee from Plant Cell Suspension Cultures” by P.M. Townsley, Department of Food Science, Faculty of Agricultural Sciences, University of British Columbia Vancouver 8, B.C. is mentioned as prior art. The article presents potential possibilities of plant cell culture in the production of coffee. After having produced the biomass containing coffee cells, they are removed from the nutrient liquid media by filtration. The excess water was absorbed by placing the cells on dry filter paper. The cells were then roasted directly by means of a roasting oven, which was a tube constructed from two semi-cylindrical ceramic electrical heating element sections. The temperature was controlled by a rheostat.

When coffee is roasted, the increased temperature and transformation of water into gas create high levels of pressure inside the beans. These conditions change the structure of the cell walls from rigid to rubbery by creating a physical change in the coffee.

The roasting step is technically problematic due to the physical differences of the green coffee bean in the traditional process versus the cell cultured coffee and any satisfying roasting process for roasting cell cultured coffee in order to produce quality coffee does not yet exist.

The object of the invention is an improved method for coffee production from cell cultured material.

### SUMMARY

The method of the invention for production of a cell-cultured ingestion product comprises the steps of providing parts of a plant and pre-processing the parts in order to obtain a cell culture by cultivation of the cells as a cell suspension in a nutrient medium. The cells are then separated from the nutrient medium and dried. The dried cells are obtained as a powder. The method is mainly characterized by pressing the obtained power into tablets.

The preferable embodiments of the method are presented in the sub claims.

The tablets are usually roasted after pressing. As was stated above, nearly all coffee sold to consumers throughout the world is sold as roasted coffee, but not necessary.

The cell culture is obtained by isolating cells from living tissue, such as from a plant, and letting the cells grow under controlled conditions. These conditions vary for each cell type, but generally they are cultivated in a suitable vessel or a bioreactor with a substrate or medium, i.e. a nutrient medium, that supplies the essential nutrients, such as sugars, vitamins, minerals, growth factors, and usually gases, like carbon dioxide CO₂, and oxygen O₂. The physio-chemical environment is regulated by adjusting e.g. pH buffer, osmotic pressure, and temperature).

The ingestion products to be produced by the method of the invention are different kinds of edible products meant to cover both drinks and foodstuffs being, however, especially meant for producing coffee or cocoa. Other roasted products from plants are: tea (Camellia sinensis); different cereal grains (like barley, oat, rye, wheat, rice, etc.), pseudocereals (like buckwheat, quinoa and chia), coffee substitutes and food additives; nuts, almonds, peanuts. These could be used in the form of cell cultures.

The plant is selected based on what ingestion product is to be produced being the coffee plant for producing coffee and the cocoa plant for producing cocoa. Tea is produced from the tea shrub, nuts from different nut trees, coffee substitutes and food additives from chicory, and cereal grains from cereals.

Also roasting is performed depending on what ingestion product is to be produced and for which use. Roasting is usually not performed for producing green coffee, tea, or cocoa or if the tablets are made from tea shrub, the cocoa plant, nut trees, chicory or cereals.

The parts of the coffee plant or cocoa plant are preferably leaves but can also be other viable parts, such as meristems or tissue, that can be transferred into the nutrient medium to constitute explants in the medium. The meristem is a type of tissue found in plants. It consists of undifferentiated cells (meristematic cells/stem cells) capable of cell division. Cells in the meristem can develop into all the other tissues and organs that occur in plants. These cells continue to divide until a time when they get differentiated and then lose the ability to divide.

The separation step in the method is preferably performed by filtration, the drying is usually performed by freeze-drying.

The pressing of the obtained powder can be performed by various equipments, such as with conventional pelletizing equipment into tablets or pellets, such as in a manual milk tablet press or manually with a screw clamp. The manual milk tablet press can be of stainless steel, such as the commercial Model Sheensy974d2tnpv, Sheens. One example of useful equipment is also a 6 Ton lab-scale pelleting machine and with no need of additional thickener. When the pelletizing is performed, manual force can be applied for the pressing.

Roasting is performed to obtain roasted tablets in a desired roasting level by adjusting the roasting time and temperature and until a change from beige colour to brown take place on both sides of the pellets, and the roasting can be performed in any conventional equipment, such as in a fan-assisted oven on a wire rack, in bags or in pressure vessels. In a small scale, it can even be performed in a pan, such as in a Teflon pan.

Thus, the invention is concerned with biotechnological production of in the first hand coffee or cocoa and comprises the step of generation of coffee or cocoa cells in bioreactors or other suitable vessels from a suspension containing the cells in a nutrient medium, separating the cells from the medium by filtration, drying the cells, with e.g. freeze-drying, thereby obtaining the cells in powder form, pelletizing the obtained powder into tablets, and optionally roasting of the tablets into a form for making the desired ingestion product, e.g. for brewing coffee.

When coffee is to be produced, the process of the invention for creating the intended type of alternative coffee begins with coffee cells, which are then placed and cultured into e.g. bioreactors to continue developing a separate biomass. Thus, the coffee is not ground from beans, but instead grown from coffee plant cells under closely controlled conditions with respect to temperature, light (most often darkness), pH and oxygen conditions in a bioreactor depending on equipment and coffee cultivar and desired properties for the end product. They are then washed with sterile water and freeze-dried.

The dried coffee cell powder is compressed into a solid structure (tablet, pellet) that resembles a conventional coffee bean. This structure can be easier roasted with conventional equipment rather than requiring completely new technical solutions to roast powder. Once roasted and ground, the product can be brewed in exactly the same way as conventional coffee.

The roasting can be performed in conventional equipment with different roasting parameters by changing the roasting duration, mixing intervals, and temperature. The resulting product can be brewed into coffee, from which the sensory properties as well as odor-active volatile compounds and caffeine contents can be determined. Conventional dark roasted coffee can e.g. be used as a reference.

Thus, the applicants have managed to produce artificial coffee through cell cultures. The resulting beans replicate natural coffee's taste and smell.

Roasting creates coffee flavor. The successful integration of a new type of material to be roasted in with existing roasting equipment and processes (with available equipment) is a key factor.

In the following the invention is described by means of certain embodiments by means of a FIGURE and embodiment examples, to the details of which the scope of protection defined by the claims are not restricted.

### FIGURE

The FIGURE is an embodiment of the method of the invention for producing coffee.

### DETAILED DESCRIPTION

An embodiment of the method of the invention is illustrated in the FIGURE.

Leaves from a coffee plant are collected 1 and young but fully developed leaves are cut off and kept at room temperature to allow for stomata closure.

A cell suspension 2 was then prepared by initiating callus on the leaves and transferring it to a medium for sub-culturing for a certain time period.

Coffee cells are then cultivated 3 in a bioreactor as a cell suspension in nutrient medium for obtaining a plant cell biomass.

The coffee cells are then separated 4 from the nutrient medium. For the separation of the coffee cells, the plant cell biomass was harvested by filtering in a Büchner funnel and washed with sterile water and then dried 5 e.g. in a freeze-dryer.

The dried coffee cells are obtained in step 6 as a powder.

The powder is pelletized 7 with a tablet machine by obtaining tablets or pellets in step 8, which remind of green coffee beans.

The tablets are then roasted 9 by using some conventional roasting equipment, such as a fan-assisted oven on wire rack, in a temperature within a range of 200-250 °C, preferably 225 °C.

When having cooled down, the roasted cell-cultured coffee tablets can be packaged 10a and then ground 11a for commerce to be brewed 12 and enjoyed 13 as ready coffee. Alternatively, the grinding can be performed before the packaging in step 11b after the grinding that instead takes place in step 10b. Thereafter, the brewing 12 can be performed and then enjoyed 13.

### Embodiment Example 1

Commercial Coffea arabica seedlings were used as a coffee plant explant source for leaves to be used for culturing coffee cells. Young but fully developed leaves were cut off and kept at room temperature to allow for stomata closure. Plates with leaves were incubated at 24 °C in darkness. Cell suspensions were established in the same medium.

For biomass production, coffee cells were cultivated in a bioreactor.

For the separation of the coffee cells, the plant cell biomass was harvested by filtering and washed with sterile water.

The washed cells were frozen and freeze dried. The freeze-dried cells were stored in a freezer until pelletizing.

The cells in powder form were pelletized for making tablets.

Roasting of the tablets was done in fan-assisted oven on wire rack, in 225 °C.

1. A method for the production of a cell-cultured ingestion product comprising the steps of:
providing parts of a plant,
pre-processing the parts in order to obtain a cell suspension,
generation of cells by cultivating as a cell suspension in a nutrient medium,
separation of the cells from the medium,
drying the cells,
obtaining the cells as a powder,
characterized by:
pressing the obtained powder into tablets.

2. The method of claim 1, wherein the tablets are roasted.

3. The method of claim 1, wherein the plant is a coffee plant, cocoa plant, tea shrub, nut tree, a cereal, or chicory.

4. The method of claim 1, wherein the parts of the plant are leaves or other tissues or meristems.

5. The method of claim 1, wherein the separation is performed by filtration.

6. The method of claim 2, wherein the roasting is performed to obtain roasted tablets in a desired roasting level by adjusting the roasting time, temperature, pH and/or light.
